(19) United States
(12) Patent Application Publication
(10) Pub. No.: US 2015/0321026 A1
(43) Pub. Date: Nov. 12, 2015

(54) DEVICES AND METHODS FOR ULTRASOUND FOCAL DEPTH CONTROL

(71) Applicant: Ulthera, Inc., Mesa, AZ (US)

(72) Inventors: Denis F. Branson, Fayetteville, NY (US); Charles D. Emery, Scottsdale, AZ (US); Joseph M Luis, Chandler, AZ (US); Michael T. Peterson, Scottsdale, AZ (US)

(21) Appl. No.: 14/405,368

(22) PCT Filed: Jun. 5, 2013
(86) PCT No.: PCT/US13/44310

§ 371 Date: Dec. 3, 2014

Related U.S. Application Data
(60) Provisional application No. 61/656,653, filed on Jun. 7, 2012.

(51) Int. Cl. A61N 7/00

(52) U.S. Cl. CPC A61N 7/00; A61N 2007/0034; A61N 2007/006

(57) ABSTRACT

Embodiments of devices, systems and methods for controlling the focal depth of energy in targeting tissue for performing various treatment and/or imaging procedures, such as cosmetic enhancement procedures. Ultrasound procedures can acoustically couple one or more spacers, offsets, standoffs, bladders, lenses, multiplexed arrays, transducer movement systems, and/or automated therapy deposition depth systems to mechanically modify a focal depth of an ultrasound transducer for treatment of tissue below tissue surface.

### CROSS-REFERENCE TO RELATED APPLICATIONS

This application is a national phase 371 application from international application PCT/US2013/044310 filed on Jun. 5, 2013, which claims the benefit of priority from U.S. Provisional Application No. 61/656,653 filed on Jun. 7, 2012, each of which are incorporated in its entirety by reference herein. Any and all priority claims identified in the Application Data Sheet, or any correction thereto, are hereby incorporated by reference under 37 CFR 1.57.

### BACKGROUND

1. Field

Embodiments of the present invention generally relate to devices, systems and methods for controlling the focal depth of energy in targeting tissue for performing various treatment and/or imaging procedures safely and effectively. Devices and methods of controlling focal depth of ultrasonic energy in cosmetic procedures are provided in several embodiments.

2. Description of the Related Art

Control of the targeting of energy delivery systems and methods to a particular depth in tissue can be performed on various forms of energy, such as acoustic, ultrasound, light, laser, radio-frequency (RF), microwave, electromagnetic, radiation, thermal, cryogenic, electron beam, photon-based, magnetic, magnetic resonance, or other energy forms. In certain instances, control of the ultrasonic focal depth can be achieved through steering or focusing ultrasound energy through mechanical and/or electrical means. For example, an aperture can mechanically focus energy at a target. Another way to change mechanical focus may be achieved by machining the transducer ceramic to a particular radius of curvature such that the waves from the aperture interfere constructively at the intended focus depth, thereby creating a specific focal gain. Some apertures can include an array of elements to electronically steer and focus the beam in multiple dimensions.

### SUMMARY

Some controls for changing focal depth involve precise machining, complex adjustment mechanism, such as translational and/or rotational motion mechanisms, and/or electronic controls. Several embodiments of the present invention are particularly advantageous because they involve a mechanical device that can be coupled to an energy source (such as an ultrasound transducer) for quick and flexible modification of focal depth with improved energy efficiency.

In some embodiments, systems and methods for diagnosis, monitoring, treatment, (e.g., cosmetic enhancement), and/or imaging of tissue with various forms of energy are intended to affect targeted tissue at one or more specific depths. In some embodiments, ultrasonic diagnosis, monitoring, therapy, treatment, cosmetic enhancement, and/or imaging procedures are intended to affect targeted tissue at a specific depth. While the acoustic energy of ultrasound treatment (e.g., cosmetic enhancement) or imaging procedures may be highly focused and localized in the targeted tissues or parts of the body, there can be a need to control, change, or modify ultrasonic focal depth. In some embodiments, it is desirable to change a focal depth to affect or to avoid certain tissues, nerves, bones, parts of the body, organs, medical devices, and/or medical implants that may or may not be intended to be treated and/or imaged from acoustic energy during these ultrasound procedures. For example, according to one embodiment, it is desirable to mechanically alter focal depth with static methods or devices.

There is a need for devices and procedures for controlling, modifying, and/or altering the focal depth of energy delivery to tissue. Various embodiments of ultrasonic focal depth control can be used to target, protect, or avoid certain tissues, body parts, organs, medical devices, and/or medical implants from acoustic energy during an ultrasound cosmetic enhancement procedure.

In one embodiment, a system for changing the focal depth of an ultrasound treatment includes an ultrasound system and an acoustic spacer. The ultrasound system includes a housing with an acoustic window and a transducer having a fixed focal depth, the transducer configured for the delivery of focused ultrasound energy to a target tissue region under a skin surface for a cosmetic improvement of the skin surface. In one embodiment, the acoustic spacer is configured for placement between the transducer and the skin surface. In one embodiment, the acoustic spacer is configured to change a focal depth of the ultrasound energy in the target tissue. In one embodiment, the acoustic spacer includes a transmitting portion that is acoustically coupled to the transducer and the skin surface. In one embodiment, the acoustic spacer is temporarily connected to the acoustic window of the housing.

In various embodiments, the acoustic spacer includes an acoustically shielding portion configured to mechanically increase a distance between the transducer and the skin surface. In one embodiment, the acoustic spacer includes a slot configured to contain an acoustic coupling agent. In one embodiment, the acoustic spacer includes a marking feature configured as a visual guide for the temporary adhesive placement of the acoustic spacer on the acoustic window of the housing. In one embodiment, the acoustic spacer includes a radio frequency identification feature configured to communicate with a software system in the ultrasound system. In one embodiment, the acoustic spacer has a uniform thickness. In one embodiment, the acoustic spacer has a variable thickness. In one embodiment, the acoustic spacer includes a variable acoustic spacer thickness that is automatically controlled through the ultrasound system. In one embodiment, the system includes a filling mechanism configured to controllably increase or decrease a volume of an acoustic coupling agent in a bladder in the acoustic spacer. In one embodiment, the transducer includes a linear multiplexed array with a series of controllable elements for changing the effective focal depth of the transducer in the target tissue region.

In one embodiment, a system for modifying the focal depth of an ultrasound treatment includes an ultrasound system and an acoustic spacer. In one embodiment, the ultrasound system includes a transducer having a fixed focal depth. In one embodiment, the transducer is configured for the delivery of focused ultrasound energy to a target tissue region under a skin surface for a cosmetic improvement of the skin surface. The acoustic spacer is configured for placement between the transducer and the skin surface. In one embodiment, the acoustic spacer is configured to mechanically increase a distance between the transducer and the skin surface. In one embodiment, the acoustic spacer comprises a transmitting portion that is acoustically coupled to the transducer and the skin surface. In one embodiment, the acoustic spacer is temporarily adhered to the skin surface. In one embodiment, the acoustic spacer is a mask configured to facilitate an ultrasonic cosmetic face lift. In one embodiment, the acoustic spacer includes an acoustically shielding portion configured to mechanically increase a distance between the transducer and the skin surface.

In one embodiment, a method for changing the focal depth of an ultrasound treatment of a region of tissue below a tissue surface includes adhering an acoustic spacer to a skin surface located proximal to a target tissue treatment region at a depth distal to the skin surface, acoustically coupling an ultrasound system to the acoustic spacer, delivering focused ultrasound at the depth in the target tissue region through said acoustic spacer; and removing the acoustic spacer from the skin surface. In one embodiment, the acoustic spacer includes a transmitting portion that is acoustically coupled to the skin surface. In one embodiment, the ultrasound system includes a transducer having a fixed focal depth. In one embodiment, the transducer is configured for the delivery of focused ultrasound energy to the target tissue region for improving a cosmetic appearance of the skin surface

In one embodiment, an acoustic depth modification device includes a transmitting region with a first and second surface separated by a transmitting region thickness that transmits at least 50% of ultrasound energy between the first and the second surface, and an adhesive layer attached to at least a portion of the first surface.

Further, areas of applicability will become apparent from the description provided herein. It should be understood that the description and specific examples are intended for purposes of illustration only and are not intended to limit the scope of the embodiments disclosed herein.

### BRIEF DESCRIPTION OF THE DRAWINGS

The drawings described herein are for illustration purposes only and are not intended to limit the scope of the present disclosure in any way. Embodiments of the present invention will become more fully understood from the detailed description and the accompanying drawings wherein:

FIG. 1A is a schematic block diagram illustrating an acoustic spacer according to various embodiments of the present invention.

FIG. 1B is a schematic illustration of an acoustic spacer and ultrasound system according to various embodiments of the present invention.

FIG. 2 is a schematic partial cut away illustration of a portion a transducer according to various embodiments of the present invention.

FIG. 3 is a partial cut away side view of an ultrasound system according to various embodiments of the present invention.

FIG. 4 is a schematic, partial cross-sectional side view of an acoustic spacer configured to attach to an ultrasound system according to an embodiment of the present invention.

FIG. 5 is a schematic, partial cross-sectional side view of an acoustic spacer configured to attach to a subject according to an embodiment of the present invention.

FIG. 6 is a schematic, partial cross-sectional side view of an acoustic spacer according to an embodiment of the present invention.

FIG. 7 is a schematic, top view of an acoustic spacer according to an embodiment of the present invention.

FIG. 8 is a schematic, top view of an acoustic spacer with a feature according to an embodiment of the present invention.

FIG. 9 is a schematic, top view of an acoustic spacer with a plurality of features according to an embodiment of the present invention.

FIG. 10 is a schematic, top view of an acoustic spacer with a plurality of features according to an embodiment of the present invention.

FIG. 11 is a schematic, cross-sectional side view of an acoustic spacer according to an embodiment of the present invention.

FIG. 12 is a schematic, cross-sectional side view of an acoustic spacer with a feature according to an embodiment of the present invention.

FIG. 13 is a schematic, cross-sectional side view of an acoustic spacer with a feature according to an embodiment of the present invention.

FIG. 14 is a schematic, cross-sectional side view of an acoustic spacer with variable thickness and a feature according to an embodiment of the present invention.

FIG. 15 is a schematic, front view of an acoustic spacer mask according to an embodiment of the present invention.

FIG. 16 is a schematic, cross-sectional side view of a bladder with a feature according to an embodiment of the present invention.

FIG. 17 is a schematic, partial cross-sectional side view of a spacer and a filling mechanism according to an embodiment of the present invention.

FIG. 18 is a schematic, cross-sectional side view of a variable spacer according to an embodiment of the present invention.

FIGS. 19A-19B are schematic, cross-sectional side views of a rotatable spacer according to an embodiment of the present invention.

FIGS. 20A-20C are schematic, partial cross-sectional side views of an ultrasound system with a lens according to various embodiments of the present invention.

FIG. 21 is a schematic, partial cross-sectional side view of an ultrasound system with a multiplexed array according to an embodiment of the present invention.

FIG. 22 is a schematic, partial cross-sectional side view of an ultrasound system with a multiplexed array according to an embodiment of the present invention.

FIGS. 23A-23D are schematic, top views of a multiplexed array according to an embodiment of the present invention.

FIG. 24 is a schematic, partial cross-sectional side view of an ultrasound system with a transducer movement system according to an embodiment of the present invention.

### DETAILED DESCRIPTION

The following description sets forth examples of embodiments, and is not intended to limit the present invention or its teachings, applications, or uses thereof. It should be understood that throughout the drawings, corresponding reference numerals indicate like or corresponding parts and features. The description of specific examples indicated in various embodiments of the present invention are intended for purposes of illustration only and are not intended to limit the scope of the invention disclosed herein. Moreover, recitation of multiple embodiments having stated features is not intended to exclude other embodiments having additional features or other embodiments incorporating different combinations of the stated features. Further, features in one embodiment (such as in one figure) may be combined with descriptions (and figures) of other embodiments.

Various embodiments of the present invention relate to devices or methods of controlling the depth of energy delivery to tissue. In various embodiments, various forms of energy can include acoustic, ultrasound, light, laser, radio-frequency (RF), microwave, electromagnetic, radiation, thermal, cryogenic, electron beam, photon-based, magnetic, magnetic resonance, and/or other energy forms. Various embodiments of the present invention relate to devices or methods of controlling ultrasonic focal depth. In various embodiments, devices or methods can be used to alter the focal depth of ultrasound in any procedures such as, but not limited to, therapeutic ultrasound, diagnostic ultrasound, non-destructive testing (NDT) using ultrasound, ultrasonic welding, any application that involves coupling mechanical waves to an object, and other procedures. Generally, with therapeutic ultrasound, a tissue effect is achieved by concentrating the acoustic energy using focusing techniques from the aperture. In some instances, high intensity focused ultrasound (HIFU) is used for therapeutic purposes in this manner. The ability to focus the power from the aperture can be described with a parameter called “focal gain” It is through this focal gain that thermal and/or mechanical ablation of tissue can occur non-invasively or remotely. The focal gain is the ratio of the aperture area to the product of the focal depth and wavelength. Therefore, a larger aperture generates a larger focal gain when compared to a smaller aperture. In general, a higher frequency (smaller wavelength) transducer generates a greater focal gain than a lower frequency transducer (larger wavelength). The focal gain gives ultrasound the ability to non-invasively treat tissues since the intensity of energy outside the focus is low enough not to significantly disturb the tissue.

In general, steering and focusing ultrasound energy may occur through mechanical and/or electrical means. In one embodiment, a control for focal depth uses an aperture to mechanically focus energy to the target. In one embodiment, the mechanical focus may be achieved by machining the ultrasonic transducer ceramic to a specific radius of curvature such that the waves from the aperture interfere constructively at the intended focus, thereby creating the necessary focal gain to affect the targeted tissue. Another way to mechanically focus the ultrasound is the use of passive materials that have different acoustic velocities relative to the target tissue where the focus occurs. In some instances, the refraction, which occurs from the velocity differences, effectively bends the ultrasound energy to the intended focus region to achieve the intended intensities.

In other embodiments, apertures can include an array of elements to electronically steer and focus the beam in three dimensions. In some embodiments, electronic delays are placed on the individual elements within the array so that the pressure wave from each element arrives at the intended focus simultaneously. In various embodiments, linear arrays, annular arrays, phased arrays, curvilinear arrays, 1.5D arrays, and 2D arrays are types of arrays that can take advantage of electronic focusing techniques.

Although the mechanical and electronic devices can create a focus in the same location, the acoustic energy deposited between the focus and the transducer is different when using mechanical as compared to electronic devices. In general, in order to reach the same intensity at the focus, electronically controlled array surface intensity is greater than the mechanically shaped transducer. This is because the arrays can have elements that are not directly pointed to the focus or a focus point. In some circumstances, an electronically controlled transducer can waste more energy due to diffraction than a mechanically focused transducer. Therefore, there can be more near-field heating (at depths in tissue between the focus and the transducer) using the electronically controlled array than a mechanically focused transducer: this can reduce the therapeutic effectiveness of the electronically controlled array. Some embodiments of the present invention address the challenges posed by using electronic arrays so ultrasonic focal depth control may be achieved with the spatial efficiency offered by a mechanically focused device. In various embodiments, mechanical focal depth adjustment devices of the present invention improve efficiency over electronic focal adjustment devices by 1%, 2%, 5%, 10%, 20%, 25%, 30%, 40%, 50%, 75%, or 100% or more. In various embodiments, the improved efficiency is in a range of roughly 1-5%, 1-10%, 1-25%, 10%-50%, 25% - 75%, and/or 25%-100% or more.

In various embodiments, a static device or method may be used to control, alter, or vary focal depth, in order to effect the formation of a lesion for a desired cosmetic and/or therapeutic treatment for a desired clinical approach at a target tissue. In various embodiments, target tissue is, but is not limited to, any of skin, eyelids, eye lash, eye brow, caruncula lacrimalis, crow's feet, wrinkles, eye, nose, mouth, tongue, teeth, gums, ears, brain, heart, lungs, ribs, abdomen, stomach, liver, kidneys, uterus, breast, vagina, prostrate, testicles, glands, thyroid glands, internal organs, hair, muscle, bone, ligaments, cartilage, fat, fat labuli, adipose tissue, subcutaneous tissue, implanted tissue, an implanted organ, lymphoid, a tumor, a cyst, an abscess, or a portion of a nerve, or any combination thereof.

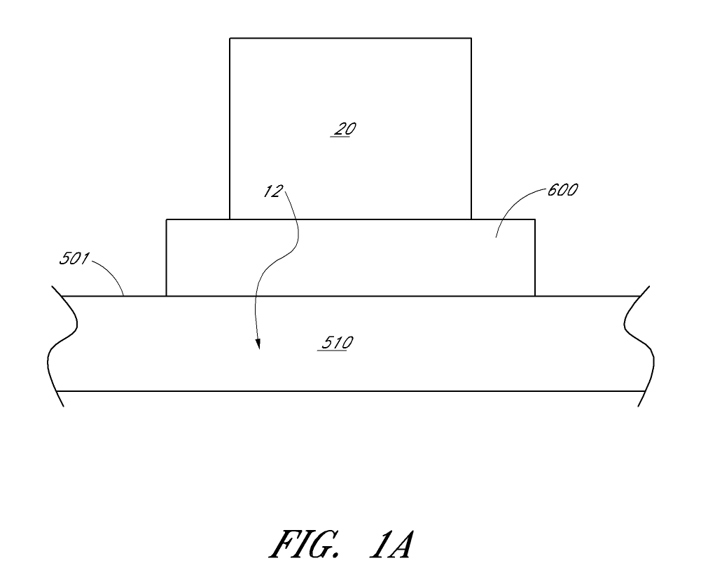

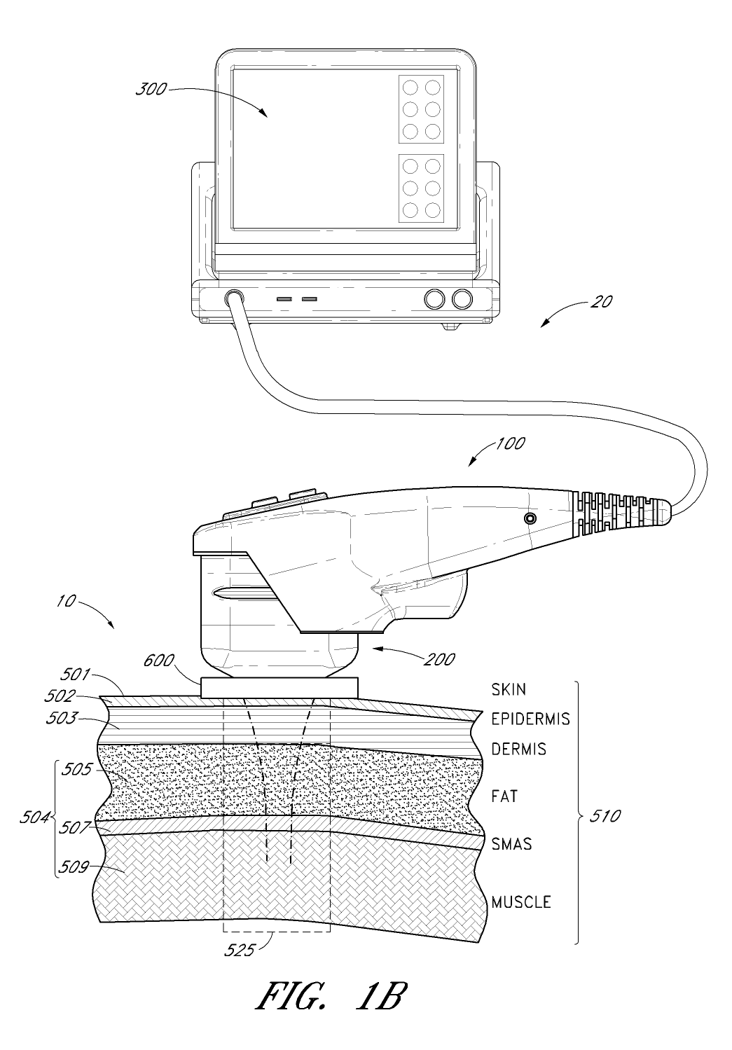

With reference to the illustration in FIG. 1A, an embodiment of an ultrasound system 20 emits energy 12 for imaging and/or treating subcutaneous tissue 510 under a skin surface 501. The ultrasound system 20 is acoustically coupled to the skin surface 501 for transmission of the energy 12 from the ultrasound system 20 to the target tissue. In one embodiment, a spacer 600 configured to control, alter, and/or vary a treatment depth of an ultrasound system 20 when placed between the ultrasound system 20 and the skin surface 501. In various embodiments, the spacer 600 is an acoustic spacer, an offset device, a standoff, a shim, a bladder, a focal depth adjustment device, a mechanical focal depth control device, and/or a focal depth modification device. In various embodiments, the acoustic spacer 600 is at least partially acoustically coupled to the ultrasound system 20, the skin surface 501, or both. In some embodiments, the spacer 600 is acoustically coupled with acoustic gel. In some embodiments, the spacer 600 is acoustically coupled with an acoustic non-gel coupling medium. In some embodiments, the spacer 600 is acoustically coupled with an acoustic gel and an acoustic non-gel coupling medium.

Various embodiments of ultrasound treatment and imaging devices are described in U.S. application Ser. No. 12/996,616, which published as U.S. Publication No. 2011-0112405 A1 on May 12, 2011, which is a U.S. National Phase under 35 U.S.C. §371 of International Application No. PCT/US2009/046475, filed on Jun. 5, 2009 and published in English on Dec. 10, 2009, which claims the benefit of priority from U.S. Provisional No. 61/059,477 filed Jun. 6, 2008, each of which is incorporated in its entirety by reference, herein. In accordance with one embodiment of the present invention, methods and systems for ultrasound treatment of tissue are configured to provide cosmetic treatment. In various embodiments of the present invention, tissue below or even at a skin surface such as epidermis, dermis, hypodermis, fascia, and superficial muscular aponeurotic system (“SMAS”), and/or muscle are treated non-invasively with ultrasound energy. Tissue may also include blood vessels and/or nerves. The ultrasound energy can be focused, unfocused or defocused and applied to a region of interest containing at least one of epidermis, dermis, hypodermis, fascia, and SMAS to achieve a therapeutic effect. FIG. 1B illustrates a schematic drawing of anatomical features of tissue layers. In various embodiments, the tissue layers can be at any part of the body of a subject 500. In one embodiment, the tissue layers are in the head and face region of a subject 500. The cross-sectional portion of tissue 10 includes a skin surface 501, an epidermal layer 502, a dermal layer 503, a fat layer 505, a superficial muscular aponeurotic system 507 (hereinafter “SMAS 507”), and a muscle layer 509. The tissue can also include the hypodermis 504, which can include any tissue below the dermal layer 503. The combination of these layers in total may be known as subcutaneous tissue 510. Also illustrated in FIG. 1B is a treatment zone 525 which is below the surface 501. In one embodiment, the surface 501 can be a surface of the skin of a subject 500. Although an embodiment directed to therapy at a tissue layer may be used herein as an example, the inventors have contemplated application of the device to any tissue in the body. In various embodiments, the device and/or methods may be used on muscles (or other tissue) of the face, neck, head, arms, legs, or any other location in the body.

With reference to the illustration in FIG. 1B, an embodiment of an ultrasound system 20 includes a hand wand 100, an emitter-receiver module 200, and a controller 300. In various embodiments, module 200 includes a transducer 280. FIG. 2 illustrates an embodiment of an ultrasound system 20 with a transducer 280 configured to treat tissue at a focal depth 278. In one embodiment, the focal depth 278 is a distance between the transducer 280 and the target tissue for treatment. In one embodiment, a focal depth 278 is fixed for a given transducer 280.

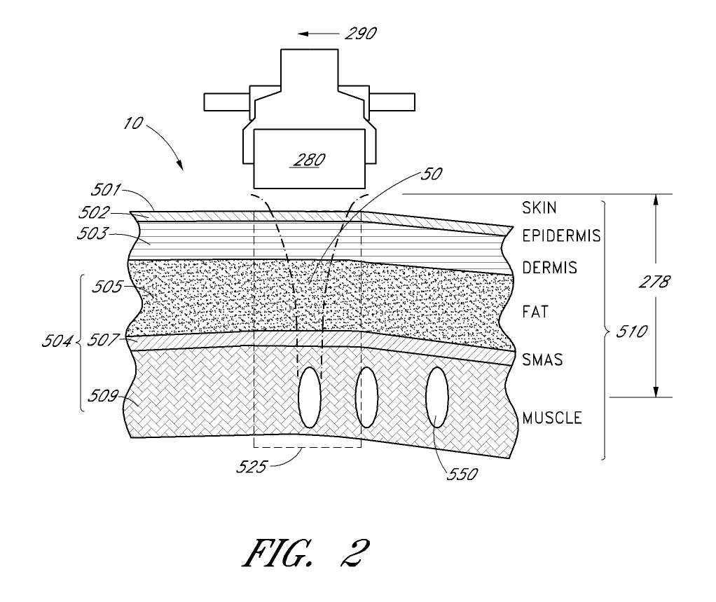

With reference to the illustration in FIG. 3, the emitter-receiver module 200 can include a transducer 280 which can emit energy through an acoustically transparent member 230. In one embodiment, the transducer 280 can have an offset distance 270, which is the distance between the transducer 280 and a surface of the acoustically transparent member 230. In various embodiments, a depth may refer to the focal depth 278. In one embodiment, the focal depth 278 of a transducer 280 is a fixed distance from the transducer. In one embodiment, a transducer 280 may have a fixed offset distance 270 from the transducer to the acoustically transparent member 230. In one embodiment, an acoustically transparent member 230 is configured at a position on the module 200 or ultrasound system 20 for contacting a skin surface 501. In various embodiments, the focal depth 278 exceeds the offset distance 270 by an amount to correspond to treatment at a target area or region of interest located at a tissue depth 279 below a skin surface 501. In various embodiments, when an ultrasound system 20 placed in physical contact with a skin surface 501, the tissue depth 279 is a distance between the acoustically transparent member 230 to the target zone, measured as the distance from the portion of the hand wand 100 or module 200 surface that contacts skin (with or without an acoustic coupling gel, medium, etc.) and the depth in tissue from that skin surface contact point to the target area. In one embodiment, the focal depth 278 can correspond to the sum of an offset distance 270 (as measured to the surface of the acoustically transparent member 230 in contact with a coupling medium and/or skin 501) in addition to a tissue depth 279 under the skin surface 501 to the target region.

Coupling components can comprise various devices to facilitate coupling of the transducer 280 or module 200 to a region of interest. For example, coupling components can comprise an acoustic coupling system configured for acoustic coupling of ultrasound energy and signals. Acoustic coupling system with possible connections such as manifolds may be utilized to couple sound into the region-of-interest, provide liquid- or fluid-filled lens focusing. The coupling system may facilitate such coupling through use of one or more coupling mediums, including air, gases, water, liquids, fluids, gels, solids, non-gels, and/or any combination thereof, or any other medium that allows for signals to be transmitted between the transducer 280 and a region of interest. In one embodiment one or more coupling media is provided inside a transducer. In one embodiment a fluid-filled emitter-receiver module 200 contains one or more coupling media inside a housing. In one embodiment a fluid-filled module 200 contains one or more coupling media inside a sealed housing, which is separable from a dry portion of an ultrasonic device. In various embodiments, a coupling medium is used to transmit ultrasound energy between one or more devices and tissue with a transmission efficiency of 100%, 99% or more, 98% or more, 95% or more, 90% or more, 80% or more, 75% or more, 60% or more, 50% or more, 40% or more, 30% or more, 25% or more, 20% or more, 10% or more, and/or 5% or more.

In various embodiments of the present invention, the transducer 280 can image and treat a region of interest at a tissue depth 279 of less than about 10 mm. In one embodiment, the emitter-receiver module 200 has a focal depth 278 for a treatment at a tissue depth 279 of about 4.5 mm below the skin surface 501 when an acoustically transparent member 230 is coupled to a skin surface 501. Some non-limiting embodiments of transducers 280 or modules 200 can be configured for delivering ultrasonic energy at a tissue depth of 3 mm, 4.5 mm, 6 mm, less than 3 mm, between 3 mm and 4.5 mm, more than more than 4.5 mm, more than 6 mm, and anywhere in the ranges of 0-3 mm, 0-4.5 mm, 0-25 mm, 0-100 mm, and any depths therein. In one embodiment, an ultrasound system 20 is provided with two transducer modules, in which the first module applies treatment at a tissue depth of about 4.5 mm and the second module applies treatment at a tissue depth of about 3 mm. An optional third module that applies treatment at a tissue depth of about 1.5-2 mm is also provided.

In various embodiments, changing the tissue depth 279 for an ultrasonic procedure is particularly advantageous because it permits treatment of a patient at varied tissue depths even if the focal depth 278 of a transducer 270 is fixed. This can provide synergistic results and maximizing the clinical results of a single treatment session. For example, treatment at multiple depths under a single surface region permits a larger overall volume of tissue treatment, which results in enhanced collagen formation and tightening. Additionally, treatment at different depths affects different types of tissue, thereby producing different clinical effects that together provide an enhanced overall cosmetic result. For example, superficial treatment may reduce the visibility of wrinkles and deeper treatment may induce formation of more collagen growth.

Although treatment of a subject at different depths in one session may be advantageous in some embodiments, sequential treatment over time may be beneficial in other embodiments. For example, a subject may be treated under the same surface region at one depth in week one, a second depth in week two, etc. The new collagen produced by the first treatment may be more sensitive to subsequent treatments, which may be desired for some indications. Alternatively, multiple depth treatment under the same surface region in a single session may be advantageous because treatment at one depth may synergistically enhance or supplement treatment at another depth (due to, for example, enhanced blood flow, stimulation of growth factors, hormonal stimulation, etc.). In several embodiments, different transducer modules provide treatment at different depths. In one embodiment, a single transducer module can be adjusted or controlled for varied depths. Safety features to minimize the risk that an incorrect depth will be selected can be used in conjunction with the single module system.

In several embodiments, a method of treating the lower face and neck area (e.g., the submental area) is provided. In several embodiments, a method of treating (e.g., softening) mentolabial folds is provided. In other embodiments, a method of treating the eye region is provided. Upper lid laxity improvement and periorbital lines and texture improvement will be achieved by several embodiments by treating at variable depths. By treating at varied depths in a single treatment session, optimal clinical effects (e.g., softening, tightening) can be achieved. In several embodiments, the treatment methods described herein are non-invasive cosmetic procedures. In some embodiments, the methods can be used in conjunction with invasive procedures, such as surgical facelifts or liposuction, where skin tightening is desired. In various embodiments, the methods can be applied to any part of the body.

In one embodiment, a transducer module permits a treatment sequence at a fixed depth at or below the skin surface. In one embodiment, a transducer module permits a treatment sequence at a fixed depth below the dermal layer. In several embodiments, the transducer module comprises a movement mechanism configured to direct ultrasonic treatment in a sequence of individual thermal lesions at a fixed focal depth. In one embodiment, the linear sequence of individual thermal lesions has a treatment spacing in a range from about 0.01 mm to about 25 mm. In one embodiment the individual thermal lesions are discrete. In one embodiment, the individual thermal lesions are overlapping. In one embodiment, the movement mechanism is configured to be programmed to provide variable spacing between the individual thermal lesions. First and second removable transducer modules are also provided. Each of the first and second transducer modules are configured for both ultrasonic imaging and ultrasonic treatment. The first and second transducer modules are configured for interchangeable coupling to a hand wand. The first transducer module is configured to apply ultrasonic therapy to a first layer of tissue, while the second transducer module is configured to apply ultrasonic therapy to a second layer of tissue. The second layer of tissue is at a different depth than the first layer of tissue.

As illustrated in FIG. 2, in various embodiments, delivery of emitted energy 50 at a suitable focal depth 278, distribution, timing, and energy level is provided by the emitter-receiver module 200 through controlled operation by the control system 300 to achieve the desired therapeutic effect of controlled thermal injury to treat at least one of the epidermis layer 502, dermis layer 503, fat layer 505, the SMAS layer 507, the muscle layer 509, and/or the hypodermis 504. Note that FIG. 2 illustrates one embodiment of a depth that corresponds to a depth for treating muscle. In various embodiments, the depth can correspond to any tissue, tissue layer, skin, epidermis, dermis, hypodermis, fat, SMAS, muscle, blood vessel, nerve, or other tissue. During operation, the emitter-receiver module 200 and/or the transducer 280 can also be mechanically and/or electronically scanned along the surface 501 to treat an extended area. In addition, spatial control of a tissue treatment depth 279 can be suitably adjusted in various ranges, such as between a wide range of about 0 mm to about 25 mm, suitably fixed to a few discrete or variable depths, with an adjustment limited to a fine range, for example, approximately between about 3 mm to about 9 mm, and/or dynamically adjusted during treatment, to treat at least one of the epidermis layer 502, dermis layer 503, hypodermis 504, fat layer 505, the SMAS layer 507 and/or the muscle layer 509. Before, during, and after the delivery of ultrasound energy 50 to at least one of the epidermis layer 502, dermis layer 503, hypodermis 504, fat layer 505, the SMAS layer 507 and/or the muscle layer 509, monitoring of the treatment area and surrounding structures can be provided to plan and assess the results and/or provide feedback to the controller 300 and the user via a graphical interface 310.

In one embodiment, an ultrasound system 20 generates ultrasound energy which is directed to and focused below the surface 501. This controlled and focused ultrasound energy creates the lesion 550 which may be a thermally coagulated zone or void in subcutaneous tissue 510. In some embodiments, the emitted energy 50 targets the tissue below the surface 501 which cuts, ablates, coagulates, micro-ablates, manipulates, and/or causes a lesion 550 in the tissue portion 10 below the surface 501 at a specified focal depth 278. In one embodiment, during the treatment sequence, the transducer 280 moves in a direction denoted by the arrow marked 290 at specified intervals 295 to create a series of treatment zones 254 each of which receives an emitted energy 50 to create one or more lesions 550.

On the face, important structures such as nerves, parotid gland, arteries and veins are present over, under or near the SMAS 507 region. Treating through localized heating of regions of the SMAS 507 layer or other suspensory subcutaneous tissue 510 to temperatures of about 60° C. to about 90° C., without significant damage to overlying or distal/underlying tissue, or proximal tissue, as well as the precise delivery of therapeutic energy to the SMAS layer 507, and obtaining feedback from the region of interest before, during, and after treatment can be suitably accomplished through the ultrasound system 20. In addition, the SMAS layer 507 varies in depth and thickness at different locations, for example from about 0.5 mm to about 5 mm or more.

In various embodiments, an acoustic spacer 600 is configured to control, alter, or vary a treatment depth of an ultrasound transducer. In one embodiment, a tissue treatment depth 279 can be altered in order to effect the formation of a lesion for a desired cosmetic and/or therapeutic treatment for a desired clinical approach. One method to obtain various foci is the use of an acoustic spacer 600. Use of one or more acoustic spacers 600 moves the focus of an ultrasound transducer to a shallower depth in tissue. This changes the location of the depth of the ultrasound treatment. For example, in one embodiment suppose the focus of the ultrasound system 20 has a fixed focal depth 278 of 10 mm and the acoustic spacer 600 has a thickness of 2 mm. The focus for the treatment zone in tissue with the spacer 600 is 10 mm minus 2 mm, or 8 mm. Any number of variations of thicknesses and materials can be used and/or combined to vary the depth according to the user's needs.

In various embodiments, the acoustic spacer 600 is a spacer, a shim, a bladder, a lens, a mask, a template, a guide, a shield, an adhesive layer, an acoustic bandage, or other device. In various embodiments, a spacer 600 may come in various fixed or variable thicknesses, in kits with one or more spacers 600. In one embodiment, a standoff may be recognizable by the system software of the ultrasound system 20 to determine the TIS (soft tissue thermal index), TIB (bone thermal index), MI (mechanical indices, a measure of acoustic power output) and ISPTA (spatial peak temporal average intensity) in tissue so appropriate transmit excitation parameters are used. In one embodiment, a spacer 600 can be identified with an RFID (radio frequency identification) tag. In one embodiment, a spacer 600 can be identified with acoustic methods. In various embodiments, the thickness of the spacer 600 can be measured, or acoustic markers and/or acoustic signatures can be used to identify the spacer 600. In one embodiment, a spacer 600 can be identified by visually such as by marking, color coding, shape and manually selecting the standoff being used so appropriate transmit excitation parameters are used. In one embodiment, the standoff may be a disposable. In one embodiment, the spacer 600 can be reused.

In various embodiments, the spacer 600 may be attached to the ultrasound system 20, attached to the skin surface 501, or free standing. With reference to FIG. 4, one embodiment of an acoustic spacer 600 is attached to the ultrasound system 20. In various embodiments, the spacer 600 is permanently attached, temporarily attached and/or removably attached to the ultrasound system 20 with an adhesive, welding, interface, locking mechanism, magnet, or other attaching device or method. In some embodiments, a coupling agent may need to be placed between the spacer 600 and a transducer 280. In some embodiments, the transducer 280 is housed in a module 200 with an acoustically transparent member 230. In some embodiments, the spacer 600 is coupled, with a coupling agent, to an acoustically transparent member 230. The spacer 600 would maintain a thickness between the acoustically transparent member 230 and a skin surface 501. In one embodiment, a coupling agent may still need to be used between the spacer 600 and the skin surface 501.

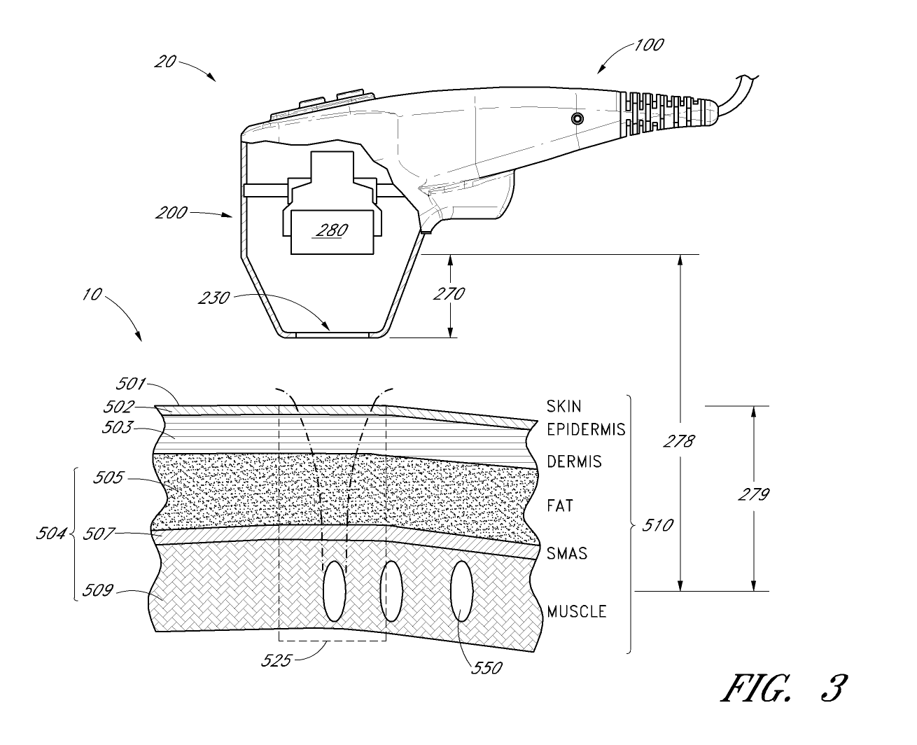

With reference to FIG. 5, one embodiment of an acoustic spacer 600 is removably or temporarily attached to a patient's skin. In one embodiment, the standoff is stiff enough such that distance from the top of the spacer 600 to the epidermis or skin surface 501 is fixed. In one embodiment, an acoustic transmitting medium or an acoustic coupling agent may be placed between the skin and the bottom of the standoff. In one embodiment, adhesion of the spacer 600 to the skin is made with one or more biocompatible adhesives on the bottom and/or sides of the spacer 600. In one embodiment, the spacer 600 is configured like tape. In one embodiment, the spacer 600 is configured like a bandage. In one embodiment, the spacer 600 includes soft silicone. In one embodiment, the spacer 600 includes fabric. In one embodiment, the spacer 600 includes a polyurethane film. In one embodiment, the spacer 600 is configured for repeated application or repositioning. In one embodiment, the spacer 600 is waterproof

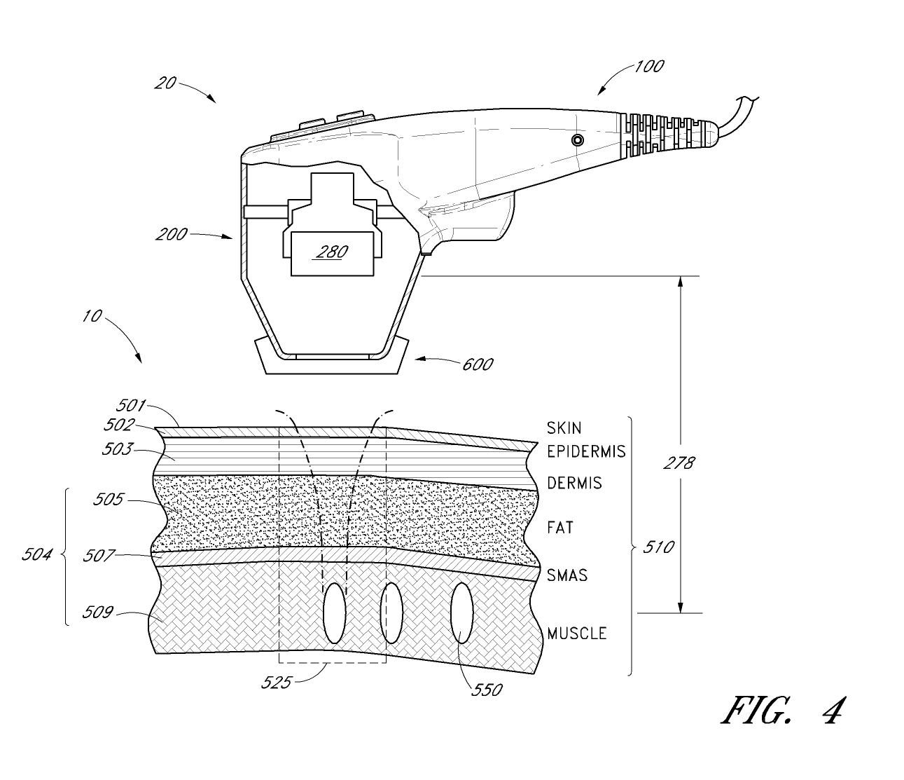

With reference to FIG. 6, one embodiment of an acoustic spacer 600 is free standing, where there is no adhesive attachment to the skin and the ultrasound system 20. In one embodiment, the position of the spacer 600 is maintained through the procedure.

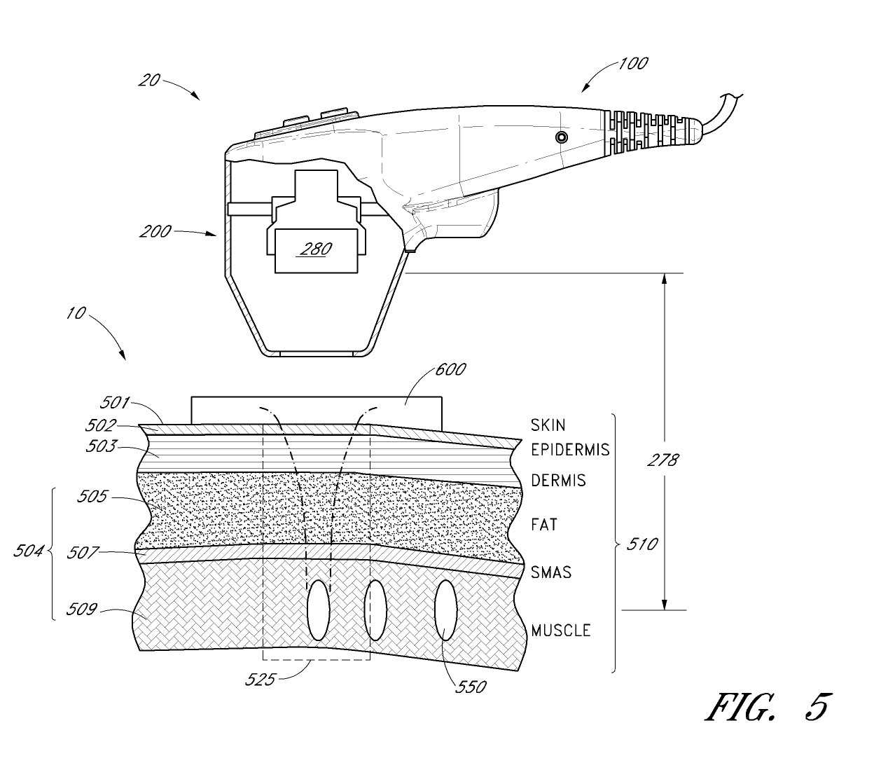

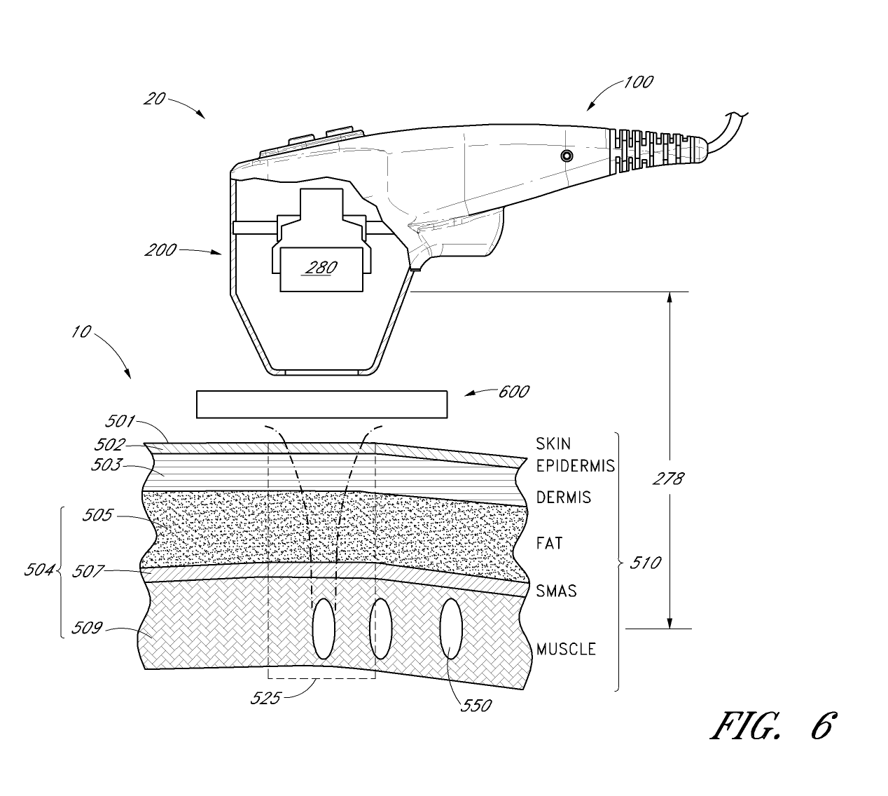

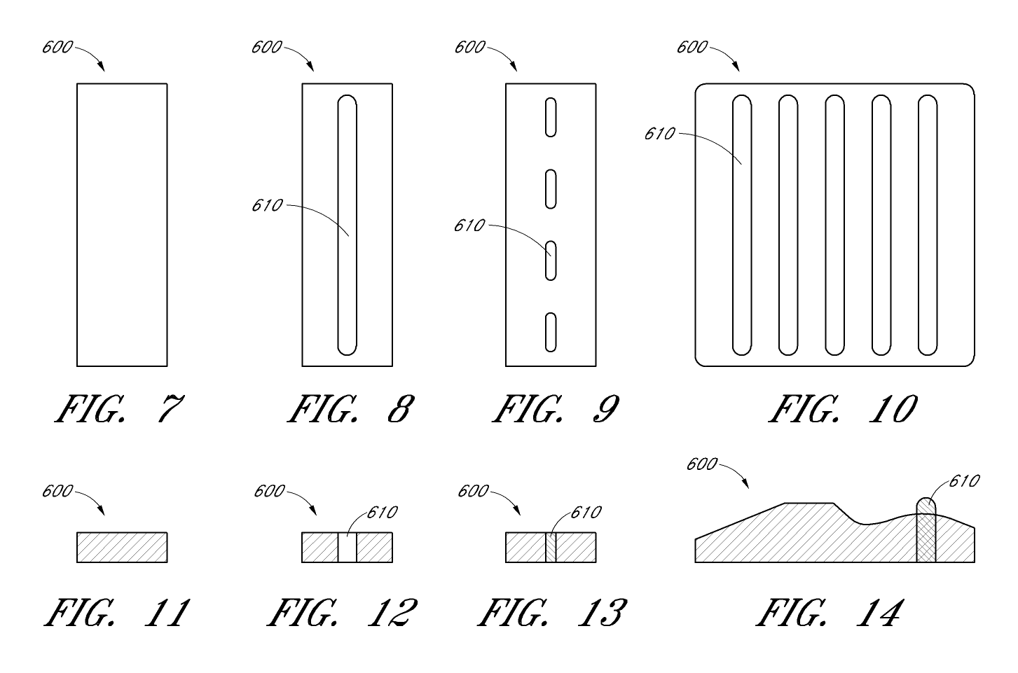

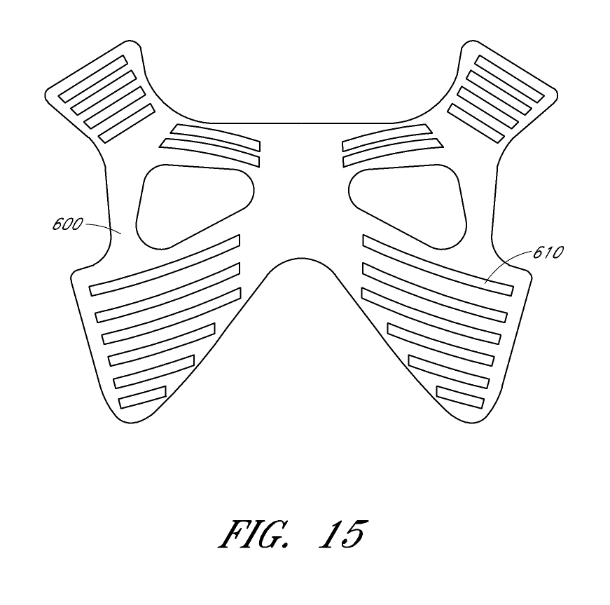

With reference to FIGS. 7-15, various embodiments of acoustic spacers 600 can include one or more materials configured for acoustic coupling transmission. In some embodiments, the spacer 600 can be used to change the path of the acoustic waves using special materials that have different velocities between the region distal to the standoff 600 and/or proximal to the standoff 600. Furthermore, in one embodiment, the spacer 600 may be shaped with this velocity-altering material to make the focus deeper or shallower than without the space 600. In one embodiment, the spacer 600 is configured for efficient acoustic coupling to minimize or reduce ultrasound energy transmission loss, for transmitting ultrasonic energy between the ultrasound system 20 and the skin. In some embodiments, the spacer 600 includes a feature 610. In one embodiment, the feature 610 has the same acoustic coupling transmission properties as the rest of the spacer 600. In one embodiment, the feature 610 has the different acoustic coupling transmission properties as the rest of the spacer 600. In one embodiment, the feature 610 is an acoustic coupling zone, for transmission of ultrasound energy. In one embodiment, the feature 610 is an acoustic shielding zone, for reducing or blocking the transmission of ultrasound energy. In one embodiment, the feature 610 is on a surface of the spacer 600. In one embodiment, the feature 610 is in the spacer 600. In one embodiment, the feature 610 is a slot or opening in the spacer 600, as shown in FIG. 12. In various embodiments, such as shown in one example in FIG. 14, the feature 610 can have a different thickness than adjacent portions of the spacer 600. In one embodiment, the feature 610 extends above the spacer 600. In one embodiment, the feature 610 extends below the spacer 600. In one embodiment, the feature 610 does not extend above or below the spacer 600. In various embodiments, a spacer 600 can include features 610 configured for delivery of a specific pattern of energy to tissue. In one embodiment, the feature 610 is a marker to guide the positioning, orientation, and/or movement of an ultrasound system 20 with respect to a procedure. In one embodiment, a feature 610 causes echoes (e.g. highly echoic compared to other regions). In one embodiment, a feature 610 is absortive, or non-reflective (e.g., anechoic compared to other regions). In one embodiment, the feature 610 is configured to guide an ultrasound system 20. In one embodiment, the feature 610 is configured to be rigid. In one embodiment, the feature 610 is configured to be compliant. In one embodiment, the feature 610 comprises a region with higher friction than another portion of the spacer 600. In one embodiment, the feature 610 comprises a region with lower friction than another portion of the spacer 600. In various embodiments, differences in friction can be used to guide the ultrasound system 20. In various embodiments, a spacer 600 may have mechanical registration with the ultrasound system 20 such that proper movement between ultrasound procedures. In one embodiment, a feature 610 mechanically registers with the ultrasound system 20 for facilitating ultrasound treatments. In one embodiment, one or more features 610 are present on a spacer 600. In one embodiment, a spacer 600 is configured with features 600 to facilitate a proper side to side spacing of treatment along a skin surface 501, such as spacing of treatment lines. In one embodiment, at least part of a spacer 600 can be used as a guide for an ultrasound system 20, such that a transducer or module can mechanically rest on the spacer 600 during a procedure. In one embodiment, a portion of a spacer 600 can remain outside the acoustic propagation path. In one embodiment, a spacer 600 or feature 610 is scalloped to give the user a sense of where to move a transducer for a procedure.

In one embodiment, the spacer 600 has uniform thickness. In one embodiment, the spacer 600 has variable thickness. Referring to FIG. 14, one embodiment of a spacer 600 can have portions with variable thickness and/or constant thickness. In various embodiments, a spacer 600 can comprise flat, sloped, tapered, curved, rounded, faceted, angular, or other shaped surfaces. In one embodiment, a spacer 600 is contoured or tapered to take into account the surface of the skin 501. In one embodiment, contouring or tapering may be done discretely. In one embodiment, the feature 610 has uniform thickness. In one embodiment, the feature 610 has variable thickness. In various embodiments, thickness can be 0.1 mm, 0.2 mm, 0.25 mm, 0.3 mm, 0.4mm, 0.5 mm, 0.6 mm, 0.7 mm. 0.75 mm, 0.8 mm, 0.9 mm, 1 mm, 1.5 mm, 2 mm, 2.5 mm, 3 mm, 3.5 mm, 4 mm, 5 mm or other thicknesses. In various embodiments, thicknesses can be in the range of 0.1-0.5 mm, 0.25-0.75 mm, 0.1-1 mm, 0.1-2 mm, 0.1-5 mm or other ranges. In various embodiments, thicknesses can be in millimeters, mils, centimeters, inches, or fractions thereof. In some embodiments, two or more spacers 600 can be used in a procedure. In one embodiment, two or more spacers 600 with same or different thicknesses and optional features 610 can be used for a procedure. In various embodiments, two or more spacers 600 can be used simultaneously or in sequence for a procedure. In various embodiments, a portion or entire spacer 600 can be spaced apart from, placed adjacent to, placed side by side with, and/or layered on top or below a second spacer 600. In various embodiments, two or more spacers 600 can be used to vary overall thickness to adjust focal depth.

In various embodiments, a spacer 600 can have any shape. In some embodiments, the spacer 600 can be square, rectangular, round, circular, oval, ellipse, triangular, and/or any polygon, cross pattern, mesh, grid, and/or pattern. In some embodiments, the spacer 600 is configured for placement on a specific part of a body. In various embodiments, the spacer 600 can be shaped or configured for procedures on the face, neck, body, body portion. In one embodiment, as shown in one non-limiting example in FIG. 15, the spacer 600 can be configured as a mask. In various embodiments, a spacer 600 can be preconfigured, modular, and/or custom shaped for a procedure or a subject.

In one embodiment, the spacer 600 is a shim. In one embodiment, the spacer 600 is a spacer. In various embodiments, a part or entire spacer 600 can placed outside of the acoustic propagation. In various embodiments, a spacer 600 can have a fixed thickness, but may vary depending on the application. In some embodiments, different spacers 600 may be applied to achieve different thicknesses or one spacer 600 may be added on top of the other spacer 600 to add an offset. In some embodiments, a coupling agent may be placed inside or where the propagation window exists. In some embodiments, one or more spacers 600 are attached by various mechanical means to either the patient or transducer.

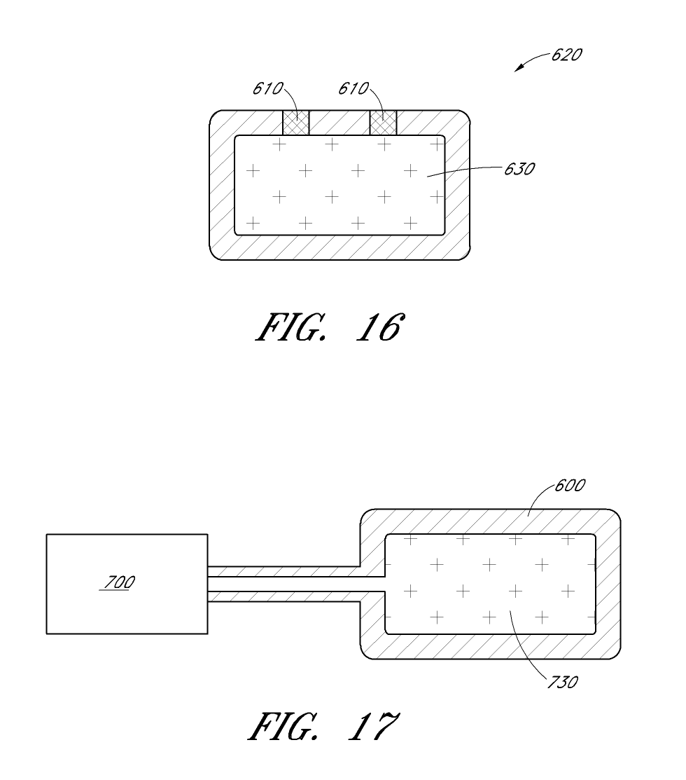

As shown in FIG. 16, in one embodiment, the spacer 600 is a bladder 620. In one embodiment, a bladder 620 is a closed vesicle or sac that would be structurally designed to add an offset between the ultrasound system 20 and the target tissue. In one embodiment, a bladder 620 is filled with an acoustic coupling agent 630. In various embodiments, a bladder 620 may be attached to the transducer or patient. In various embodiments, a bladder 620 may optionally include one or more features 610.

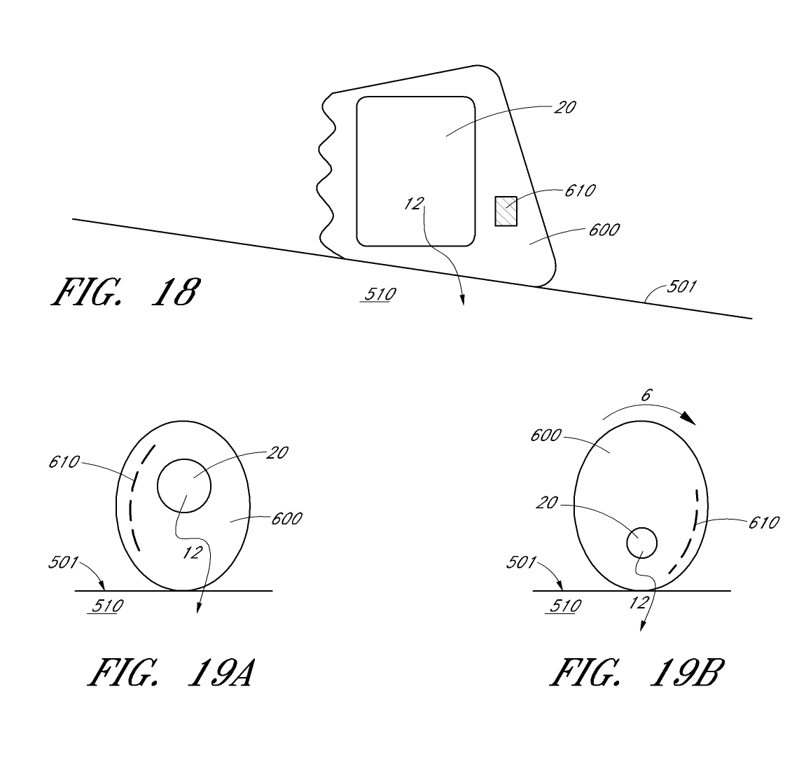

FIG. 17 illustrates one embodiment of a filling mechanism 700 that may be used to either manually or automatically change the thickness of a spacer 600 or a bladder 620. In one embodiment, a filling mechanism 700 is a closed flow system where a filler medium 730 is either removed or added to change the thickness of the at least a portion of the spacer 600. In one embodiment, different media 730 can be introduced in to the spacer 600 to alter the path of the acoustic energy, which can alter focal depth. In one embodiment, a filling mechanism 700 can be used to compress the tissue so the variable or various treatment depths are achieved. In various embodiments, a filling mechanism may be used to pinch, compress, alter, move, spread out, flatten, stretch or manipulate tissue for a procedure. In one embodiment, a filling mechanism 700 comprises a disposable cartridge of acoustic medium, which can be used to continuously fill a chamber in the spacer 600. In one embodiment, a filling mechanism 700 is configured to vary dynamically based on a target tissue. In one embodiment, a filling mechanism 700 monitors the target tissue and can vary dynamically dependent on the type of target tissue. In one embodiment, a filling mechanism 700 can monitor a sensor or reference to dynamically vary the thickness of a spacer 600 or bladder 630. In one embodiment, a filling mechanism 700 is configured to adjust the temperature of a spacer 600 or bladder 630. In one embodiment, a filling mechanism 700 monitors the temperature of a spacer 600 or bladder 630. In one embodiment, a filling mechanism 700 monitors and/or adjusts the temperature of a tissue surface in contact with a spacer 600 or a bladder 630.

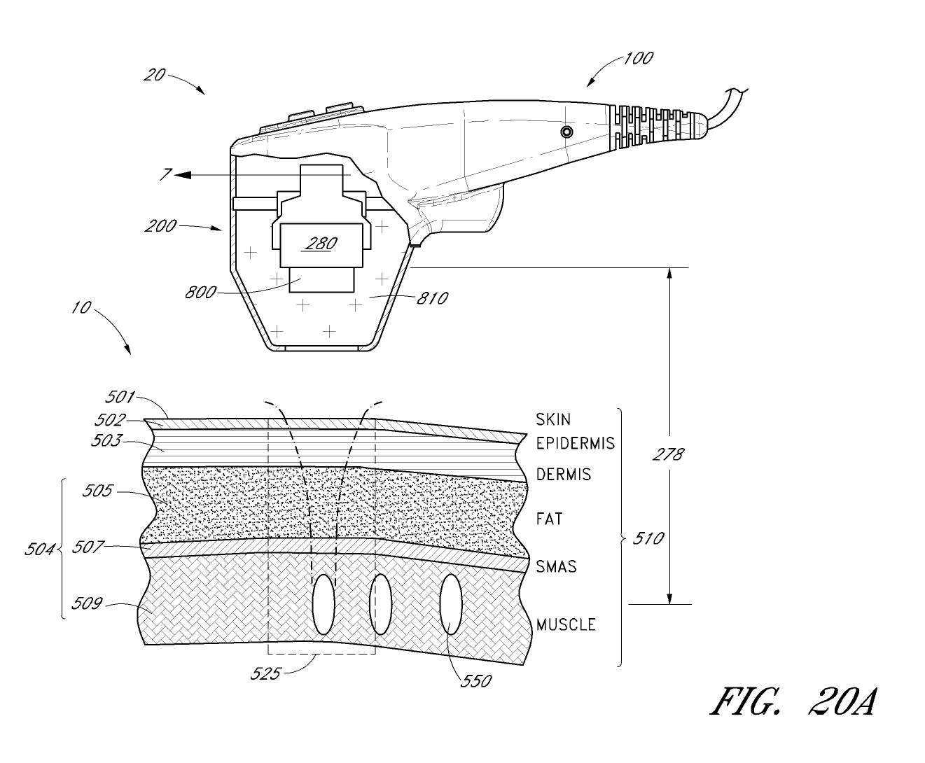

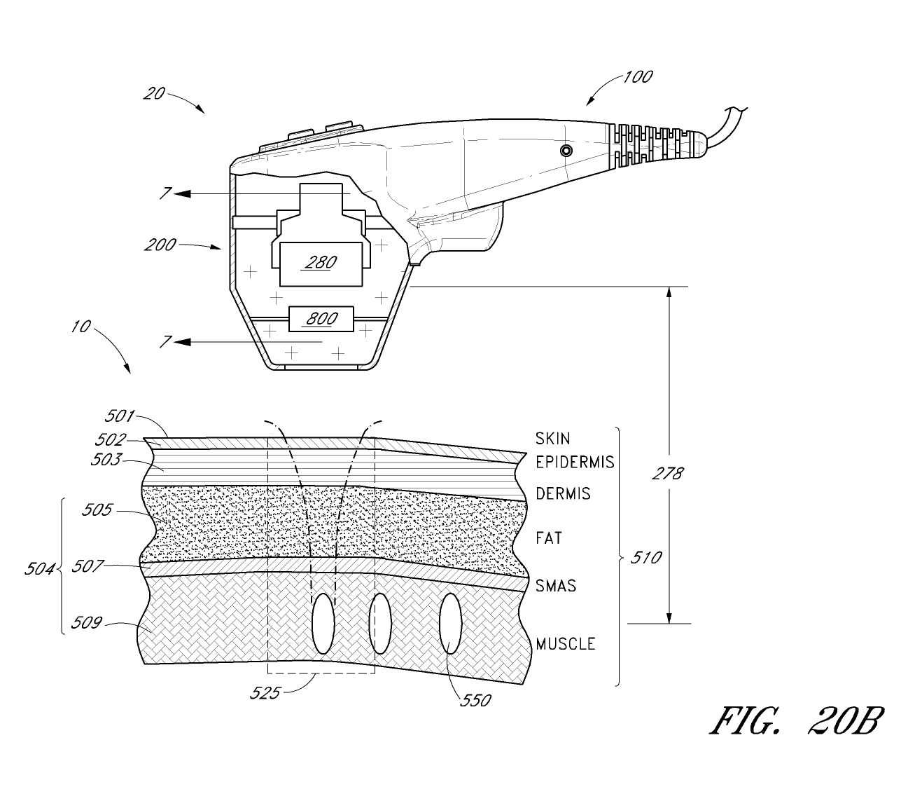

As shown in FIGS. 18 and 19A-19B, in various embodiments, the spacer 600 is a mechanical moveable ring or band around the transducer of the ultrasound system 20 that can be rotated or moved manually or automatically to change a focal depth in delivering energy 12 to a target tissue. In one embodiment, the spacer 600 has moves up and down automatically. Based on the transducer orientation, the spacer 600 may move up and down such that each thermal dose is at the same tissue depth. As shown in FIGS. 19A-19B, one embodiment of a rotatable spacer 600 can change the depth of energy 12 delivery to a tissue by rotating 6 to vary spacer 600 thickness.

In various embodiments, the thickness of a spacer 600 can be measured. In one embodiment, the thickness of a spacer 600 is measured using software through analysis of an RF pulse used to locate a reflective surface (e.g. no energy is reflected back to the transducer until the ultrasound hits the bottom surface of the standoff) In one embodiment, the thickness of a spacer 600 is measured using software through image recognition One or more features 610 may include reflective targets may be added to the spacer 600 to assist users during treatment in alignment of the device for a procedure. In one embodiment, reflective targets would not significantly modify the acoustic beam. In one embodiment, software may be used to recognize one or more features 610 for performing a procedure. In one embodiment, software used to recognize one or more features 610 as markers may be used for guiding a procedure or treatment with monitoring of position and feedback to a user. In one embodiment, measurement of the thickness of a spacer 600 can be used for altering or controlling the filling of a spacer 600 or bladder 620.

In various embodiments, a lens 800 can be used to generate different mechanical foci. In one embodiment, a lens 800 is separate from a spacer 600. In one embodiment, a lens 800 is part of a spacer 600. In one embodiment, a lens 800 comprises a compound lens. In various embodiments, an acoustic lens 800 may be used to alter a focus depth. In some embodiments, an ultrasound system 20 can include a focused ceramic bowl transducer. In some embodiments, an ultrasound system 20 can include a lens 800 that is placed between a transducer and the target tissue to produce a focus in the tissue. In one embodiment, a transducer may comprise a piezoelectric ceramic that is flat, and an acoustic lens 800 is placed between the transducer and target tissue.

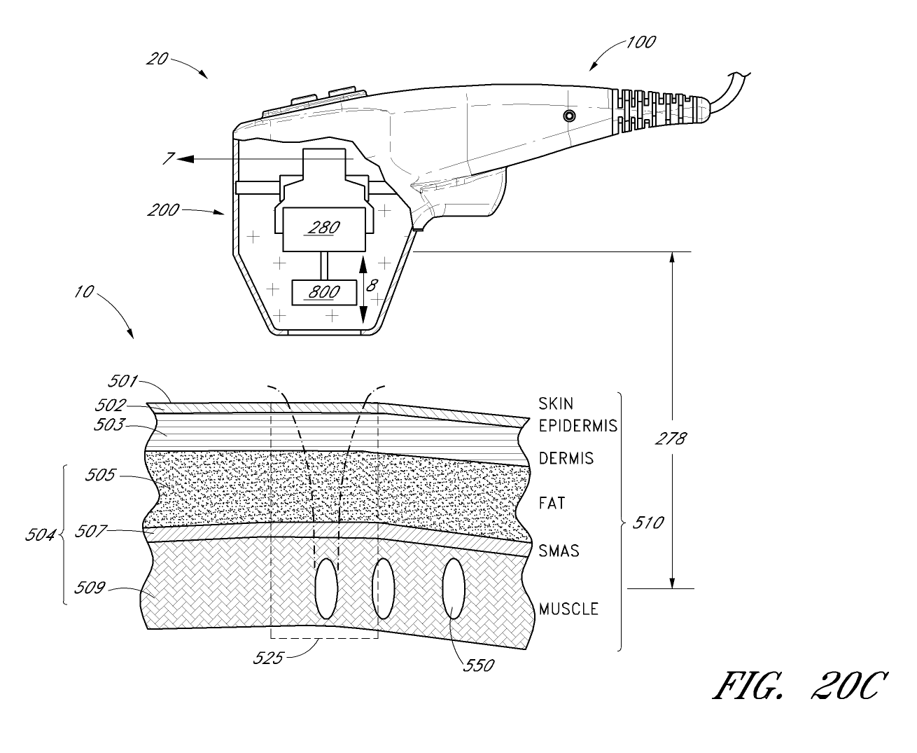

In one embodiment, as shown in FIG. 20A, a lens 800 and transducer 280 are submerged in an acoustic coupling agent 810 inside a module 200 and move synchronously in a direction 7 such that the axis of the lens 800 coincides with the axis of the transducer 280. In various embodiments, a focal depth 278 location in tissue may be moved by changing the lens 800 that is between the transducer 280 and patient. In various embodiments, one, two, three, or more lenses 800 can be used with a transducer 280. In one embodiment, a focal depth location in tissue may be moved by placing an additional lens 800 in front of or behind an existing acoustic lens 800. In various embodiments, adding or removing lenses 800 to the transducer 280 is done manually or automatically using control mechanisms in the transducer module 200 or ultrasound system 20. In one embodiment, a compound lens system moves synchronously with a piezoelectric disc transducer 280. In one embodiment, a lens 800 moves independently of a transducer 820. In one embodiment, as shown in FIG. 20B, a lens 800 is configured to move parallel to a transducer 280 in a direction 7. In various embodiments, the lens 800 can move synchronously, in unison, asynchronously, and/or independently of the transducer 280. In one embodiment, a lens 800 is configured to move perpendicular to a transducer 280 in a direction 8. In one embodiment, as shown in FIG. 20C, a lens 800 is configured to move parallel and perpendicular to a transducer 280. In one embodiment, a lens 800 is configured to move with respect to a transducer 280 to change a focal depth 278 of the transducer 270 with any embodiment of a movement system. In some embodiments, the shape of the transducer 280 may also be selectable to further change a depth of treatment.

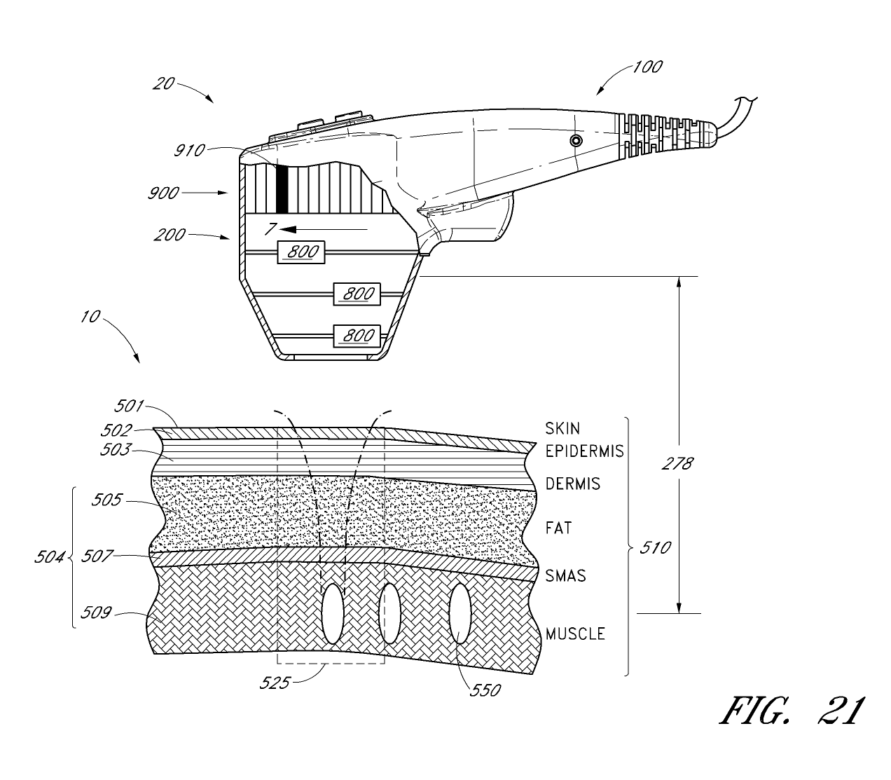

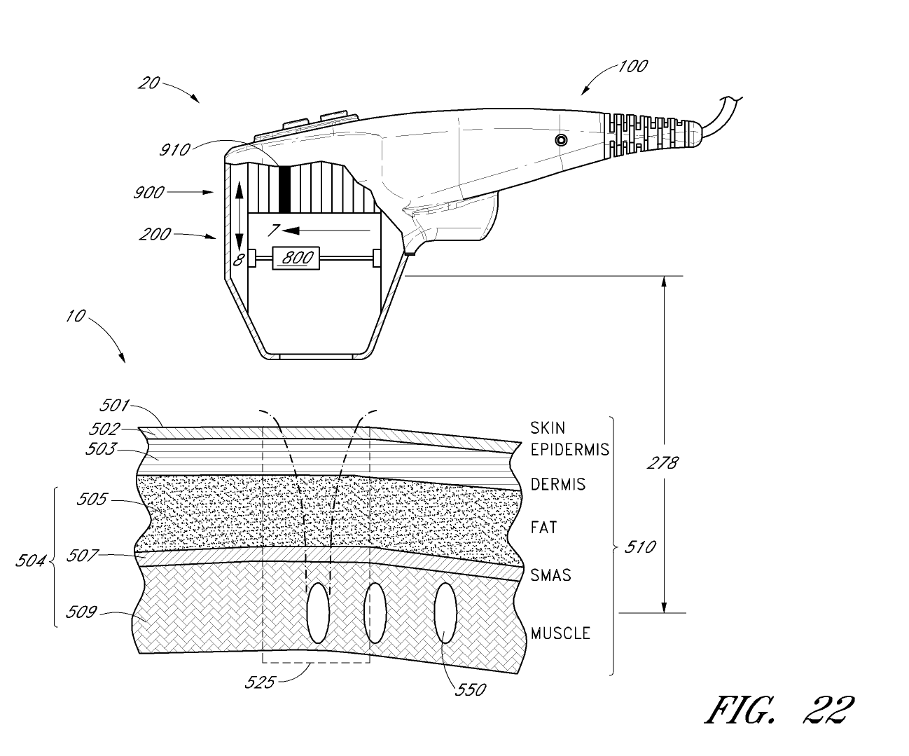

In various embodiments, a multiplexed array 900 can be used to generate different mechanical foci. In one embodiment, a multiplex array 900 is a transducer. In one embodiment, a multiplex array 900 is a multi-element transducer. In one embodiment, a multiplexed array 900 is linear. In one embodiment, a multiplex array 900 is a transducer that spans the treatment length of a module 200. In various embodiments, a multiplexed array 900 operates with one or more lenses 800. In one embodiment, a multiplexed array 900 maintains a fixed or stationary position within a module 200, while activating or moving one or more array elements 910 along the multiplexed array 900. In one embodiment, a multiplexed array 900 is located between a transducer 280 and target tissue. In one embodiment, a multiplexed array 900 selectively activates one or more elements 910 in a direction 7 in a module 200. In one embodiment, as shown in FIGS. 21 and 22, a multiplexed array 900 is configured to delivery ultrasonic energy as a transducer in a transducer module 200. In one embodiment, one lens 800 is configured to move in a direction 7 in the module 200. In various embodiments, one, two, three, or more lenses 800 can be moved in any direction 7, 8 with respect to any element 910 in a multiplexed array 900. In various embodiments, the position and/or combination of one or more lenses 800 alters the focal depth 278 of a procedure. In one embodiment, the movement or position of an active element 910 is synchronized with a position of one or more lenses 800.

In various embodiments, the multiplexed array 900 has any number of elements 910 that can be excited or activated at any one time. In various embodiments, a multiplexed array 900 can have any element 910 activated or deactivated in any sequence at any time. In one embodiment, elements 910 are controlled to produce a sequence or pattern. In embodiment, the multiplexed array 900 comprises a series of mechanically separated piezoelectric elements that are electrically connected to one or more electronic switches. In one embodiment, the elements are connected to one or more channels with one or more excitation transmission circuits. In various embodiments, one or more elements are active and one or more elements are inactive. In one embodiment, the elements to not have any time delays. In one embodiment, a single excitation circuit is tied to a fixed number of active elements 910. In various embodiments, zero, one, two, three, four, five, six, seven, eight, nine, ten, or more elements 910 are active at a time.

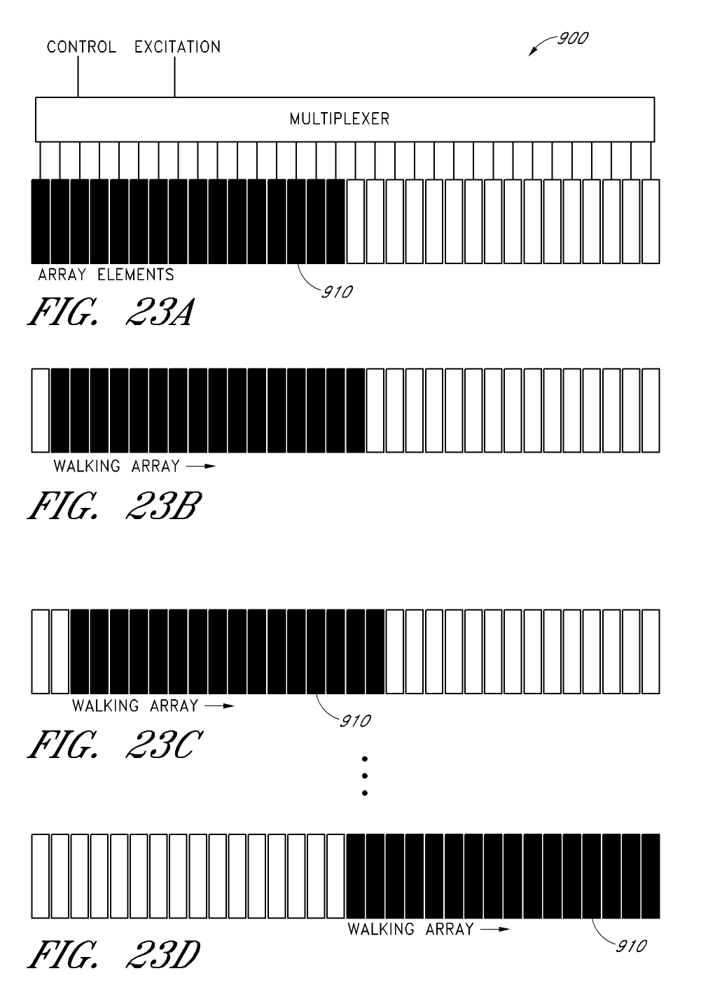

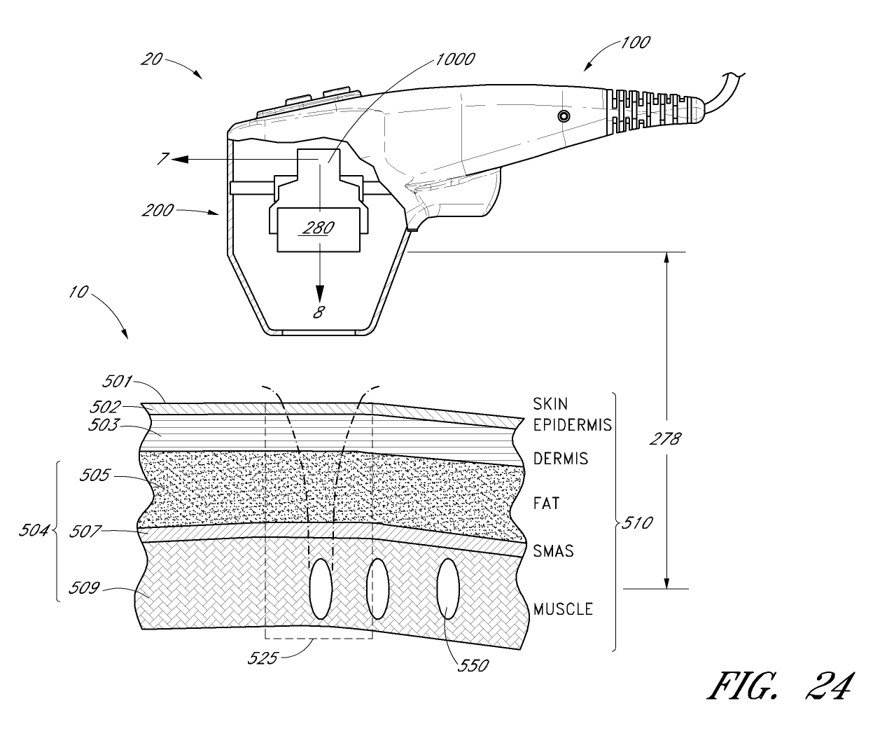

For example, FIGS. 23A-23D show one embodiment of a sequence of element 910 activation with a 32 element multiplexed array 900 that allows up to 16 elements to be excited at one time. In various embodiments, an aperture corresponding to active elements 910 can move from left to right and/or right to left electronically to vary the elements 910 excited. In one embodiment, a time delay is applied to the individual array elements 910 to vary the focus. In one embodiment, there is no delay placed on the individual array elements 910 to vary the focus. In one embodiment, a flat focus or uniform delay is place on the elements 910. In one embodiment, a flat focus or uniform delay is place on the elements 910 with one transmit excitation. In various embodiments, the array 900 may be shaped to give additional energy 12 beam control.

In one embodiment, interchangeable lenses 800 are placed in front of the multiplexed array 900. The multiplexing is synchronized with the lens 800 movement such that the energy 12 beam axis coincides with the lens 800 center axis. In one embodiment, using a stationary multiplexed array 900 decreases the amount of mass moved by a motor.

As shown in FIG. 24, in various embodiments, a transducer movement system 1000 can be used to generate different mechanical foci. Multiple focal depths can be achieved by mechanically moving the transducer 280 and relevant focusing components using an actuator. In addition to having an actuator move the transducer in a direction 7 parallel to the surface of the skin so multiple thermal depositions can be made along a focal depth, an actuator may move the transducer and relevant components in a direction 8 up, down, closer or farther from the skin surface to adjust the treatment depth.

In various embodiments, an automated therapy deposition depth system 1100 can be used to generate different foci. In some embodiments, an electronic controlled array uses delays to control the depth of the focus. In either the mechanical or electronic case, if the proposed device has imaging capabilities (e.g. B-mode) or A-mode capabilities, the system may automatically adjust the transmitted foci based on the contours of the tissue (e.g., such as the skin, skin layers, a face, etc.) to create a lesion at the same depth for the entire treatment line. For example, in one embodiment, an ultrasound system 20 places twenty-five lesions in a line. In certain cases, contours of the treated tissue, thickness of the soft tissue and position of the bone may cause significant variation of the lesion depth in tissue. The ultrasound image or RF ultrasound pulse may be used to determine the distance between the treatment depth and the transducer aperture which eliminates the need for the treatment area to be of uniform thickness. In one embodiment, an automated therapy deposition depth system 1100 automatically varies the depth of each lesion deposition to place the lesion at a prescribed depth in tissue. In various embodiments, the automated therapy deposition depth system 1100 can change the depth by filling or removing coupling agent from a bladder 620, changing height of a spacer 600, moving a lens 800, using other depth control techniques, and/or any combination. In one embodiment, an automated therapy deposition depth system 1100 automatically adjusts depth to account for changes in pressure of applying an ultrasound system 20 against a tissue. The pliability of the tissue may change the distance of the transducer from the treatment depth. An automatic electronic and/or mechanical focusing system could automatically adjust for this movement immediately prior to, or during treatment.

There are several advantages to use of embodiments of the systems and methods disclosed herein. Various techniques, systems and devices reduce the need for multiple transducers and gives greater flexibility to the user within one device. For example, various spacers 600 can include guides that help the user to properly execute procedures, such as, e.g. registering treatment lines and movements to a next line of treatment. Use of various embodiments of spacers 600, lenses 800, arrays 900, transducer movement systems 1000, and/or automated therapy deposition depth systems 1100 help maintain the focal efficiency from the mechanically focused transducer 280 and gives depth control without using multiple devices during treatment. In some embodiments, recognition, measuring and/or monitoring of specific devices reduces or prevents human error in treatment selection. In some embodiments, recognition, measuring and/or monitoring of specific devices reduces or prevents pain during procedures. In various embodiments, a filling mechanism 700 maintains the coupling agent in the treatment area such that there is no need to pick up a transducer 280 and reapply acoustic coupling agent. In various embodiments, a compound lens 800 is fully enclosed to give multiple depth treatments and reduces motor torque requirements. Use of some embodiments of the systems and methods reduce or eliminate the formation of air bubble from forming on a surface of the transducer 280 or ultrasound system 20.

Some embodiments and the examples described herein are examples and not intended to be limiting in describing the full scope of compositions and methods of these invention. Equivalent changes, modifications and variations of some embodiments, materials, compositions and methods can be made within the scope of the present invention, with substantially similar results.

1. A system for changing the focal depth of an ultrasound treatment, comprising:
an ultrasound system comprising a housing with an acoustic window and a transducer having a fixed focal depth, the transducer configured for the delivery of focused ultrasound energy to a target tissue region under a skin surface for a cosmetic improvement of the skin surface; and
an acoustic spacer configured for placement between the transducer and the skin surface,
wherein the acoustic spacer is configured to change a focal depth of the ultrasound energy in the target tissue,
wherein the acoustic spacer comprises a transmitting portion that is acoustically coupled to the transducer and the skin surface; and
wherein the acoustic spacer is temporarily connected to the acoustic window of the housing.

2. The system according to claim 1, the acoustic spacer further comprising an acoustically shielding portion configured to mechanically increase a distance between the transducer and the skin surface.

3. The system according to claim 1, the acoustic spacer further comprising a slot, the slot configured to contain an acoustic coupling agent.

4. The system according to claim 1, wherein the acoustic spacer further comprises a marking feature configured as a visual guide for the temporary adhesive placement of the acoustic spacer on the acoustic window of the housing.

5. The system according to claim 1, wherein the acoustic spacer further comprises a radio frequency identification feature configured to communicate with a software system in the ultrasound system.

6. The system according to claim 1, wherein the acoustic spacer further comprises a uniform thickness.

7. The system according to claim 1, wherein the acoustic spacer further comprises a variable thickness.

8. The system according to claim 1, wherein a variable acoustic spacer thickness is automatically controlled through the ultrasound system.

9. The system according to claim 1, further comprising a filling mechanism configured to controllably increase or decrease a volume of an acoustic coupling agent in a bladder in the acoustic spacer.

10. The system according to claim 1, wherein the transducer comprises a linear multiplexed array with a series of controllable elements for changing the effective focal depth of the transducer in the target tissue region.

11. A system for modifying the focal depth of an ultrasound treatment, comprising:
an ultrasound system comprising a transducer having a fixed focal depth, the transducer configured for the delivery of focused ultrasound energy to a target tissue region under a skin surface for a cosmetic improvement of the skin surface; and
an acoustic spacer configured for placement between the transducer and the skin surface,
wherein the acoustic spacer is configured to mechanically increase a distance between the transducer and the skin surface,
wherein the acoustic spacer comprises a transmitting portion that is acoustically coupled to the transducer and the skin surface,
wherein the acoustic spacer is temporarily adhered to the skin surface.

12. The system of claim 11, wherein the acoustic spacer is a mask configured to facilitate an ultrasonic cosmetic face lift.

13. The system of claim 11, the acoustic spacer further comprising an acoustically shielding portion configured to mechanically increase a distance between the transducer and the skin surface.

14. The system of claim 11, the acoustic spacer further comprising a slot, the slot configured to contain an acoustic coupling agent.

15. The system of claim 11, wherein the acoustic spacer further comprises a marking feature configured as a visual guide for the placement of the acoustic window over said skin surface.

16. The system of claim 11, wherein the acoustic spacer further comprises a radio frequency identification feature configured to communicate with a software system in the ultrasound system.

17. The system of claim 11, wherein the acoustic spacer further comprises a uniform thickness.

18. The system of claim 11, wherein the acoustic spacer further comprises a variable thickness.

19. The system of claim 11, further comprising a filling mechanism configured to controllably increase or decrease a volume of an acoustic coupling agent in a bladder in the acoustic spacer.

20. The system of claim 11, wherein the transducer comprises a linear multiplexed array with a series of controllable apertures for changing the effective focal depth of the transducer in the target tissue region.

21. (canceled)

22. (canceled)
